(19) United States
(12) Patent Application Publication
(10) Pub. No.: US 2005/0069597 A1
(43) Pub. Date: Mar. 31, 2005

(54) Method of using a compound of menthol and L-arginine as a preparation for the topical delivery of alprostadil for the treatment of female sexual dysfunction

(76) Inventors: Ronald J. Thompson, Ft. Thomas, KY (US); James M. Thompson, Cincinnati, OH (US); Justin R. Thompson, Cincinnati, OH (US)

Correspondence Address: Donald N. Halgren, 35 Central Street, Manchester, MA, 01944, US

(21) Appl. No.: 10/989,978

(22) Filed: Nov. 16, 2004

Related U.S. Application Data
(63) Continuation-in-part of application No. 10/803,148, filed on Mar. 17, 2004.
(63) Continuation-in-part of application No. 10/731,692, filed on Dec. 9, 2003.
Application No. 10/731,692 is a division of application No. 10/004,091, filed on Oct. 23, 2001, now Pat. No. 6,702,733.
Application No. 10/004,091 is a continuation of application No. 09/520,110, filed on Mar. 7, 2000, now Pat. No. 6,322,493.
Application No. 09/520,110 is a continuation-in-part of application No. 09/469,959, filed on Dec. 21, 1999, now abandoned.
Application No. 09/469,959 is a continuation-in-part of application No. 09/414,250, filed on Oct. 7, 1999, now Pat. No. 6,224,541.
Application No. 09/414,250 is a continuation-in-part of application No. 09/340,227, filed on Jul. 1, 1999, now Pat. No. 6,179,775.

(51) Int. Cl. A61K 31/198; A61K 35/78

(52) U.S. Cl. 424/739; 424/747; 424/742; 514/565; 424/745

(57) ABSTRACT

A manual application of a topical clitoral sensitizing compound combination consisting og L-arginine and a cooling agent comprised of Menthol and aplrostadil, wherein the combination is applicable manually to the clitoris

### BACKGROUND OF THE INVENTION

1. Field of the Invention

This invention relates to arrangements for the stimulation of females and more particularly to topical application of specialized stimulatory medicaments. This application is a continuation-in-part application of co-pending U.S. patent applications Ser. Nos. 10/803,148 and. 10/731,692, both filed 9 Dec. 2003 which are divisional applications of U.S. patent application Ser. No. 10/004,091, filed 23 Oct. 2001, now U.S. Pat. No. 6,702,733 issued 9 Mar. 2004, which is a continuation of application Ser. No. 09/520,110, filed 7 Mar. 2000, now U.S. Pat. No. 6,322,493 which is a continuation-in-part of application Ser. No. 09/469,959 filed on 21 Dec. 1999, which is a continuation-in-part of application Ser. No. 09/414,250, filed on 7 Oct. 1999, now U.S. Pat. No. 6,224,541 which is a continuation-in-part of application Ser. No. 09/340,227, filed on 1 Jul. 1999, now U.S. Pat. No. 6,179,775 each of which are incorporated herein by reference in their entirety.

2. Prior Art

U.S. Pat. Nos. 6,322,493 and 6,702,733 describe a combination of menthol and L-arginine, topically applied to the mucous membrane of the female clitoris, to increase vasodilatation of the corpus cavernosa. The menthol initially causes vasodilatation and secondarily functions as a lipophilic vehicle to enhance the clitoral absorption of L-arginine. The increased tissue concentrations of L-arginine cause induction of the nitric oxide synthase pathway, production of nitric oxide and cyclic-GMP, for the persistent vasodilation of the corpus cavernosa.

patent application Ser. No. 10/803,148, filed Mar. 17, 2004, incorporated herein by reference, entitled: “A method of using a compound of menthol and L-arginine as a preparation for the topical delivery of a 5-phosphodiesterase Inhibitor for the Treatment of female sexual dysfunction” takes this treatment further. The invention recited therein describes a treatment method whereby using the menthol component of our compound also as a vehicle for the enhanced topical absorption of a 5-phosphodiesterase inhibitor.

### BRIEF SUMMARY OF THE INVENTION

The present invention relates to the substitution of a potent vasodilator, prostaglandin E1, its synthetic preparations, such as alprostadil, and analogs thereof, for the 5-phosphodiesterase inhibitor, recited in the aforementioned patent application Ser. No. 10/803,148, in combination with topically applied menthol and L-arginine for the treatment of female sexual dysfunction.

Thus, the instant invention is a combination of three vasodilators, menthol, L-arginine, and alprostadil, all with different methods of action for inducing vasodilation. This unique arrangement permits the reducing of the dosage of each component while still achieving effectiveness of corpus cavemosa enlargement and clitoral sensitivity and rigidity. The menthol component of this proposed combination would also act as a vehicle for the absorption of the alprostadil. The vasodilatory properties of the alprostadil would enhance the absorption of L-arginine. L-arginine also insures a sufficient amount of substrate for supply of the nitric oxide synthase system for the production of nitric oxide and cyclic GMP, the vasodilator products of the nitric oxide synthase pathway. Therefore, the previously un-described combination of menthol/L-arginine and aprostadil presents a multitude of novel synergistic interactions between and amongst the individual components that would be much more effective in the treatment of female sexual dysfunction, and at much lower concentrations of each of the components.

Alprostadil is available as an intraurethral suppository (for men only), and as an injectable solution to be directly injected into the male corpus cavemosum. They are both available as prescription medication, effective for the treatment of male impotence, or erectile dysfunction. MUSE and CAVERJECT are the trademark names for these two therapies for male impotence, respectively.

### DETAILED DESCRIPTION OF THE PRESENT INVENTION

The present invention relates to a manually applicable topical preparation comprising a compound of menthol or a menthol substitute, and L-arginine, and alprostadil as that topical compound. This manually applicable compound is to be manually applied directly to a woman's clitoris, for the treatment of female sexual dysfunction. Such alprostadil in one preferred embodiment may preferably comprise less than 10% of that topical compound.

A first preferred embodiment of a Menthol substitute may be peppermint oil. A second preferred embodiment of a Menthol substitute may be cornmint oil. A third preferred embodiment of a Menthol substitute may be Eucalyptus oil. A fourth preferred embodiment of a Menthol substitute may be Citronella oil. A fifth preferred embodiment of a Menthol substitute may be Camphor oil. A sixth preferred embodiment of a Menthol substitute may be Cinnamon oil. A seventh preferred embodiment of Menthol would include Menthol analogs and derivatives including: (+)-neo-Menthol, Menthone, (+)-iso-Menthone, Menthyl acetate, Menthyl isovalerate, (−)-Menthyl lactate, para-Menth-1-en-3-ol, Piperitone, (−)-Menthol ethylene glycol carbonate, (−)Menthol 1- and 2-propylene glycol carbonate, (−)-Mentholel 1,2-glycerol ketal, (+)-Menthonel 1,2-glycerol ketal, and mono-Methyl succinate.

The invention thus comprises a manual application of a topical clitoral sensitizing combination consisting essentially of L-arginine and a cooling agent comprised of Menthol and alprostadil, wherein the combination is applicable manually to the clitoris. The combination in one preferred embodiment is contained in a hand manipulable reservoir or container to facilitate direct application to the clitoris, or to the hand of the person for such hand application to the clitoris.

The cooling agent may in various preferred embodiments include peppermint oil. The cooling agent may include cornmint oil. The cooling agent may include Eucalypus oil. The cooling agent may include Citronella oil. The cooling agent may include Camphor oil. The cooling agent may include Cinnamon oil. The cooling agent may essentially comprise peppermint oil. The cooling agent may essentially comprise cornmint oil. The cooling agent may essentially comprise Eucalyptus oil. The cooling agent may essentially comprise Citronella oil. The cooling agent may essentially comprise Camphor oil. The cooling agent essentially may comprise Cinnamon oil. The combination in one preferred embodiment may contain less than 10% L-arginine. The combination in another preferred embodiment may contain less than 10% menthol and the combination may contain less than 10% L-arginine. The combination in a further preferred embodiment may contain less than 10% alprostadil.

The topical sensitizing combination may include an analog of Menthol such as for example:
a. (+)-neo-Menthol
b. Menthone
c. (+)-iso-Menthone
d. Menthyl acetate
e. Menthyl isovalerate
f. (−)-Menthyl lactate
g. para-menth-1-en-3ol
h. Piperitone
i. (−)-Menthol ethylene glycol carbonate
j. (−)-Menthol 1-and 2-propylene glycol carbonate
k. (−)-Menthone 1,2-glycerol ketal
l. (+)-Menthone 1,2-glycerol ketal
m. mono-Menthyl succinate

1. A manual application of a topical clitoral sensitizing combination consisting essentially of L-arginine and a cooling agent comprised of Menthol and alprostadil, wherein said combination is applicable manually to the clitoris.

2. The topical sensitizing combination as recited in claim 1, wherein said cooling agent includes peppermint oil.

3. The topical sensitizing combination as recited in claim 1, wherein said cooling agent includes cornmint oil.

4. The topical sensitizing combination as recited in claim 1, wherein said cooling agent includes Eucalypus oil.

5. The topical sensitizing combination as recited in claim 1, wherein said cooling agent includes Citronella oil.

6. The topical sensitizing combination as recited in claim 1, wherein said cooling agent includes Camphor oil.

7. The topical sensitizing combination as recited in claim 1, wherein said cooling agent includes Cinnamon oil.

8. The topical sensitizing combination as recited in claim 1, wherein said cooling agent essentially comprises peppermint oil.

9. The topical sensitizing combination as recited in claim 1, wherein said cooling agent essentially comprises cornmint oil.

10. The topical sensitizing combination as recited in claim 1, wherein said cooling agent essentially comprises Eucalyptus oil.

11. The topical sensitizing combination as recited in claim 1, wherein said cooling agent essentially comprises Citronella oil.

12. The topical sensitizing combination as recited in claim 1, wherein said cooling agent essentially comprises Camphor oil.

13. The topical sensitizing combination as recited in claim 1, wherein said cooling agent essentially comprises Cinnamon oil.

14. The topical sensitizing combination as recited in claim 1, wherein said combination contains less than 10% L-arginine.

15. The topical sensitizing combination as recited in claim 1, wherein said combination contains less than 10% menthol and said combination contains less than 10% L-arginine and said combination contains less than 10% 5-phosphodiaesterase inhibitor.

16. The topical sensitizing combination as recited in claim 1, wherein said Menthol comprises an analog of Menthol.
